(19) United States
(12) Patent Application Publication
(10) Pub. No.: US 2019/0088162 A1
(43) Pub. Date: Mar. 21, 2019

(54) VIRTUAL AND/OR AUGMENTED REALITY TO PROVIDE PHYSICAL INTERACTION TRAINING WITH A SURGICAL ROBOT

(71) Applicant: Covidien LP, Mansfield, MA (US)

(72) Inventor: Dwight Meglan, Westwood, MA (US)

(73) Assignee: Covidien LP, Mansfield, MA (US)

(21) Appl. No.: 16/082,162

(22) PCT Filed: Mar. 3, 2017
(86) PCT No.: PCT/US17/20572

§ 371 Date: Sep. 4, 2018

Related U.S. Application Data
(60) Provisional application No. 62/333,309, filed on May 9, 2016.
(60) Provisional application No. 62/303,460, filed on Mar. 4, 2016.

(51) Int. Cl. G09B 23/28; A61B 34/30; G09B 9/00; G06T 19/00

(52) U.S. Cl. CPC G09B 23/28; A61B 34/30; A61B 2034/102; G06T 19/006; G09B 9/00

(57) ABSTRACT

Disclosed are systems, devices, and methods for training a user of a robotic surgical system including a surgical robot using a virtual or augmented reality interface, an example method comprising localizing a three-dimensional (3D) model of the surgical robot relative to the interface, displaying or using the aligned view of the 3D model of the surgical robot using the virtual or augmented reality interface, continuously sampling a position and orientation of a head of the user as the head of the user is moved, and updating the pose of the 3D model of the surgical robot based on the sampled position and orientation of the head of the user.

### CROSS REFERENCE TO RELATED APPLICATIONS

This application claims the benefit of U.S. Provisional Patent Application Ser. No. 62/303,460, filed Mar. 4, 2016 and U.S. Provisional Patent Application Ser. No. 62/333,309, filed May 9, 2016, the entire contents of each of which are incorporated by reference herein.

### BACKGROUND

Robotic surgical systems are increasingly becoming an integral part of minimally-invasive surgical procedures. Generally, robotic surgical systems include a surgeon console located remote from one or more robotic arms to which surgical instruments and/or cameras are coupled. A user provides inputs to the surgeon console, which are communicated to a central controller that translates the inputs into commands for telemanipulating the robotic arms, surgical instruments, and/or cameras during the surgical procedure.

As robotic surgical systems are very complex devices, the systems can present a steep learning curve for users who are new to the technology. While traditional classroom- and demonstration-type instruction may be used to train new users, this approach may not optimize efficiency as it requires an experienced user to be available to continually repeat the demonstration.

### SUMMARY

The present disclosure addresses the aforementioned issues by providing methods for using virtual and/or augmented reality systems and devices to provide interactive training with a surgical robot.

Provided in accordance with an embodiment of the present disclosure is a method of training a user of a surgical robotic system including a surgical robot using a virtual reality interface. In an aspect of the present disclosure, the method includes generating a three-dimensional (3D) model of the surgical robot, displaying a view of the 3D model of the surgical robot using the virtual reality interface, continuously sampling a position and orientation of a head of the user as the head of the user is moved, and updating the displayed view of the 3D model of the surgical robot based on the sampled position and orientation of the head of the user.

In a further aspect of the present disclosure, the method further includes tracking movement of an appendage of the user, determining an interaction with the 3D model of the surgical robot based on the tracked movement of the appendage of the user, and updating the displayed view of the 3D model of the surgical robot based on the interaction.

In another aspect of the present disclosure, the method further includes displaying commands based on a lesson plan using the virtual reality interface.

In a further aspect of the present disclosure, the method further includes determining whether the interaction corresponds to the commands, and displaying updated commands based on the lesson plan when it is determined that the interaction corresponds to the commands.

In another aspect of the present disclosure, the displaying commands include displaying commands instructing the user to perform a movement to interact with the 3D model of the surgical robot.

In yet another aspect of the present disclosure, the lesson plan includes commands instructing the user to perform actions to set up the surgical robot.

In a further aspect of the present disclosure, the method further includes displaying a score based on objective measures of proficiency used to assess a user performance based on the interactions instructed by the commands.

In another aspect of the present disclosure, the displaying includes displaying the view of the 3D model using a head-mounted virtual reality display.

In yet another aspect of the present disclosure, the displaying includes projecting the view of the 3D model using a projector system.

Provided in accordance with an embodiment of the present disclosure is a system for training a user of a surgical robotic system including a surgical robot. In an aspect of the present disclosure, the system includes a surgical robot, a virtual reality interface, and a computer in communication with the virtual reality interface. The computer is configured to generate a three-dimensional (3D) model of the surgical robot, display a view of the 3D model of the surgical robot using the virtual reality interface, continuously sample a position and orientation of a head of the user as the head of the user is moved, and update the displayed view of the 3D model of the surgical robot based on the sampled position and orientation of the head of the user.

In another aspect of the present disclosure, the computer is further configured to track movement of an appendage of the user, determine an interaction with the 3D model of the surgical robot based on the tracked movement of the appendage of the user, and update the displayed view of the 3D model of the surgical robot based on the interaction.

In a further aspect of the present disclosure, the system further includes one or more sensors configured to track the movement of the appendage of the user.

In another aspect of the present disclosure, the system further includes one or more cameras configured to track the movement of the appendage of the user.

In yet another aspect of the present disclosure, the computer is further configured to display commands based on a lesson plan using the virtual reality interface.

In a further aspect of the present disclosure, the computer is further configured to, determine whether the interaction corresponds to the commands, and display updated commands based on the lesson plan when it is determined that the interaction corresponds to the commands.

In yet a further aspect of the present disclosure, the commands instruct the user to perform a movement to interact with the 3D model of the surgical robot.

In another aspect of the present disclosure, the lesson plan includes commands instructing the user to perform actions to set up the surgical robot.

In a further aspect of the present disclosure, the computer is further configured to display a score based on objective measures of proficiency used to assess user performance based on the interactions instructed by the commands.

In another aspect of the present disclosure, includes displaying the view of the 3D model using a head-mounted virtual interface.

In yet another aspect of the present disclosure, the displaying includes projecting the view of the 3D model using a projector system.

Provided in accordance with an embodiment of the present disclosure is a non-transitory computer-readable storage medium storing a computer program for training a user of a surgical robotic system including a surgical robot. In an aspect of the present disclosure, the computer program includes instructions which, when executed by a processor, cause the computer to generate a three-dimensional (3D) model of the surgical robot, display a view of the 3D model of the surgical robot using the virtual reality interface, continuously sample a position and orientation of a head of the user as the head of the user is moved, and update the displayed view of the 3D model of the surgical robot based on the sampled position and orientation of the head of the user.

In a further aspect of the present disclosure, the instructions further cause the computer to track movement of an appendage of the user, determine an interaction with the 3D model of the surgical robot based on the tracked movement of the appendage of the user, and update the displayed view of the 3D model of the surgical robot based on the interaction.

In another aspect of the present disclosure, the instructions further cause the computer to display commands based on a lesson plan using the virtual reality interface.

In a further aspect of the present disclosure, the instructions further cause the computer to determine whether the interaction corresponds to the commands, and display updated commands based on the lesson plan when it is determined that the interaction corresponds to the commands.

In another aspect of the present disclosure, the commands instruct the user to perform a movement to interact with the 3D model of the surgical robot.

In yet another aspect of the present disclosure, the lesson plan includes commands instructing the user to perform actions to set up the surgical robot.

In a further aspect of the present disclosure, the instructions further cause the computer to display a score based on objective measures of proficiency used to assess user performance based on the interactions instructed by the commands.

In another aspect of the present disclosure, the displaying includes displaying the view of the 3D model using a head-mounted virtual interface.

In a further aspect of the present disclosure, the displaying includes projecting the view of the 3D model using a projector system.

Provided in another aspect of the present disclosure is a method of training a user of a robotic surgical system including a surgical robot using an augmented reality interface including an augmented reality interface device. The method includes detecting an identifier in an image including a physical model, matching the identifier with a three-dimensional surface geometry map of a physical model representing the surgical robot, displaying an augmented reality view of the physical model, continuously sampling a position and orientation of a user's head relative to a location of the physical model, and updating the displayed augmented reality view of the physical model based on the sampled position and orientation of the head of the user.

In another aspect of the present disclosure, the method further comprises tracking movement of an appendage of the user, determining an interaction with the physical model representing the surgical robot based on the tracked movement of the appendage of the user, and updating the displayed augmented reality view of the physical model based on the interaction.

In a further aspect of the present disclosure, the method further comprises displaying commands based on a lesson plan using the augmented reality interface.

In another aspect of the present disclosure, the method further comprises determining whether the interaction corresponds to the commands, and displaying updated commands based on the lesson plan in response to a determination that the interaction corresponds to the commands.

In a further aspect of the present disclosure, the displaying commands includes displaying commands instructing the user to perform a movement to interact with the physical model representing the surgical robot.

In yet a further aspect of the present disclosure, the lesson plan includes commands instructing the user to perform actions to set up the surgical robot.

In a further aspect of the present disclosure, the displaying includes displaying the augmented reality view of the physical model using a head-mounted augmented reality display.

In another aspect of the present disclosure, the physical model is the surgical robot.

Provided in another aspect of the present disclosure is a method of training a user of a robotic surgical system including a surgical robot using an augmented reality interface including an augmented reality interface device. The method includes detecting an identifier in an image including the surgical robot, matching the identifier with a three-dimensional surface geometry map of the surgical robot, displaying an augmented reality view of an image of the surgical robot, continuously sampling a position and orientation of the augmented reality interface device relative to a location of the surgical robot, and updating the displayed augmented reality view of the surgical robot based on the sampled position and orientation of the augmented reality interface device.

In another aspect of the present disclosure, the method further includes tracking movement of an appendage of the user, determining an interaction with the surgical robot based on the tracked movement of the appendage of the user, and updating the displayed augmented reality view of the surgical robot based on the interaction.

In a further aspect of the present disclosure, the method further includes displaying commands based on a lesson plan using the augmented reality interface.

In another aspect of the present disclosure, the method further includes determining whether the interaction corresponds to the commands, and displaying updated commands based on the lesson plan in response to a determination that the interaction corresponds to the commands.

In a further aspect of the present disclosure, the displaying commands includes displaying commands instructing the user to perform a movement to interact with the surgical robot.

In yet a further aspect of the present disclosure, the lesson plan includes commands instructing the user to perform actions to set up the surgical robot.

In a further aspect of the present disclosure, the displaying includes displaying the augmented reality view of an image of the surgical robot using a tablet, smartphone, or projection screen.

Any of the above aspects and embodiments of the present disclosure may be combined without departing from the scope of the present disclosure.

### BRIEF DESCRIPTION OF THE DRAWINGS

Various aspects and features of the present disclosure are described hereinbelow with references to the drawings, wherein:

FIG. 1 is a simplified diagram of an exemplary robotic surgical system including an interactive training user interface in accordance with an embodiment of the present disclosure;

FIG. 2 is a block diagram of a controller implemented into the robotic surgical system of FIG. 1, in accordance with an embodiment of the present disclosure;

FIG. 3 is a flow chart of a method of training a user of the robotic surgical system, in accordance with an embodiment of the present disclosure;

FIG. 4 is a flow chart of a method of training a user of the robotic surgical system, in accordance with another embodiment of the present disclosure; and

FIG. 5 is a flow chart of training a user of the robotic surgical system, in accordance with still another embodiment of the present disclosure.

### DETAILED DESCRIPTION

The present disclosure is directed to devices, systems, and methods for using virtual and/or augmented reality to provide training for the operation of a robotic surgical system. To assist a technician, clinician, or team of clinicians (collectively referred to as “clinician”), in training to configure, setup, and operate the robotic surgical system, various methods of instruction and/or use of virtual and/or augmented reality devices may be incorporated into the training to provide the clinician with physical interaction training with the robotic surgical system.

Detailed embodiments of such devices, systems incorporating such devices, and methods using the same are described below. However, these detailed embodiments are merely examples of the disclosure, which may be embodied in various forms. Therefore, specific structural and functional details disclosed herein are not to be interpreted as limiting but merely as a basis for the claims and as a representative basis for allowing one skilled in the art to variously employ the present disclosure in virtually any appropriately detailed structure.

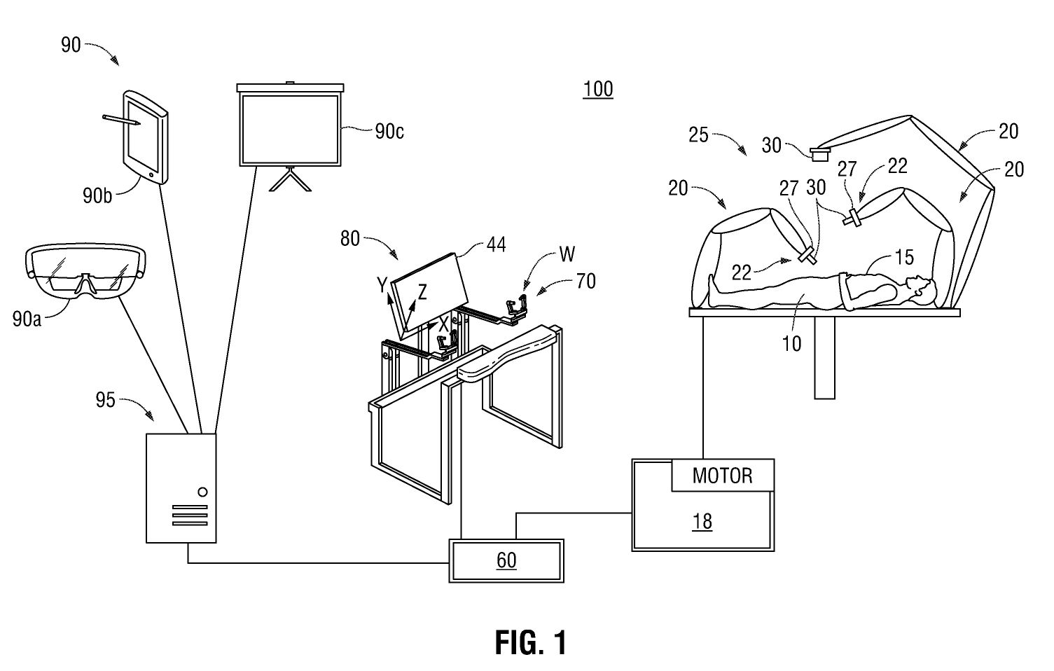

With reference to the drawings, FIG. 1 shows a robotic surgical system 100 which may be used for virtual and/or augmented reality training, provided in accordance with an embodiment of the present disclosure. Robotic surgical system 100 generally includes a surgical robot 25, a plurality of cameras 30, a console 80, one or more interactive training (IT) interfaces 90, a computing device 95, and a controller 60. Surgical robot 25 has one or more robotic arms 20, which may be in the form of linkages, having a corresponding surgical tool 27 interchangeably fastened to a distal end 22 of each robotic arm 20. One or more robotic arms 20 may also have fastened thereto a camera 30, and each arm 20 may be positioned about a surgical site 15 around a patient 10. Robotic arm 20 may also have coupled thereto one or more position detection sensors (not shown) capable of detecting the position, direction, orientation, angle, and/or speed of movement of robotic arm 20, surgical tool 27, and/or camera 30. In some embodiments, the position detection sensors may be coupled directly to surgical tool 27 or camera 30. Surgical robot 25 further includes a robotic base 18, which includes the motors used to mechanically drive each robotic arm 20 and operate each surgical tool 27.

Console 80 is a user interface by which a user, such as an experienced surgeon or clinician tasked with training a novice user, may operate surgical robot 25. Console 80 operates in conjunction with controller 60 to control the operations of surgical robot 25. In an embodiment, console 80 communicates with robotic base 18 through controller 60 and includes a display device 44 configured to display images. In one embodiment, display device 44 displays images of surgical site 15, which may include images captured by camera 30 attached to robotic arm 20, and/or data captured by cameras 30 that are positioned about the surgical theater, (for example, a camera 30 positioned within surgical site 15, a camera 30 positioned adjacent patient 10, and/or a camera 30 mounted to the walls of an operating room in which robotic surgical system 100 is used). In some embodiments, cameras 30 capture visual images, infra-red images, ultrasound images, X-ray images, thermal images, and/or any other known real-time images of surgical site 15. In embodiments, cameras 30 transmit captured images to controller 60, which may create three-dimensional images of surgical site 15 in real-time from the images and transmits the three-dimensional images to display device 44 for display. In another embodiment, the displayed images are two-dimensional images captured by cameras 30.

Console 80 also includes one or more input handles attached to gimbals 70 that allow the experienced user to manipulate robotic surgical system 100 (e.g., move robotic arm 20, distal end 22 of robotic arm 20, and/or surgical tool 27). Each gimbal 70 is in communication with controller 60 to transmit control signals thereto and to receive feedback signals therefrom. Additionally or alternatively, each gimbal 70 may include control interfaces or input devices (not shown) which allow the surgeon to manipulate (e.g., clamp, grasp, fire, open, close, rotate, thrust, slice, etc.) surgical tool 27 supported at distal end 22 of robotic arm 20.

Each gimbal 70 is moveable to move distal end 22 of robotic arm 20 and/or to manipulate surgical tool 27 within surgical site 15. As gimbal 70 is moved, surgical tool 27 moves within surgical site 15. Movement of surgical tool 27 may also include movement of distal end 22 of robotic arm 20 that supports surgical tool 27. In addition to, or in lieu of, a handle, the handle may include a clutch switch, and/or one or more input devices including a touchpad, joystick, keyboard, mouse, or other computer accessory, and/or a foot switch, pedal, trackball, or other actuatable device configured to translate physical movement from the clinician to signals sent to controller 60. Controller 60 further includes software and/or hardware used to operate the surgical robot, and to synthesize spatially aware transitions when switching between video images received from cameras 30, as described in more detail below.

IT interface 90 is configured to provide an enhanced learning experience to the novice user. In this regard, IT interface 90 may be implemented as one of several virtual reality (VR) or augmented reality (AR) configurations. In an embodiment using virtual reality (VR), IT interface 90 may be a helmet (not shown) including capabilities of displaying images viewable by the eyes of the novice user therein, such as implemented by the Oculus Rift. In such an embodiment, a virtual surgical robot is digitally created and displayed to the user via IT interface 90. Thus, a physical surgical robot 25 is not necessary for training using virtual reality.

In another VR embodiment, IT interface 90 includes only the display devices such that the virtual surgical robot and/or robotic surgical system is displayed on projection screen 90c or a three-dimensional display and augmented with training information. Such implementation may be used in conjunction with a camera or head mounted device for tracking the user's head pose or the user's gaze.

In an embodiment using augmented reality AR, IT interface 90 may include a wearable device 90a, such as a head-mounted device. The head-mounted device is worn by the user so that the user can view a real-world surgical robot 25 or other physical object through clear lenses, while graphics are simultaneously displayed on the lenses. In this regard, the head-mounted device allows the novice user while viewing surgical robot 25 to simultaneously see both surgical robot 25 and information to be communicated relating to surgical robot 25 and/or robotic surgical system 100. In addition, IT interface 90 may be useful either while viewing the surgical procedure performed by the experienced user at console 80 and may be implemented in a manner similar to the GOOGLE® GLASS® or MICROSOFT® HOLOLENS® devices.

In another augmented reality embodiment, IT interface 90 may additionally include one or more screens or other two-dimensional or three-dimensional display devices, such as a projector and screen system 90c, a smartphone, a tablet computer 90b, and the like, configured to display augmented reality images. For example, in an embodiment where IT interface 90 is implemented as a projector and screen system 90c, the projector and screen system 90c may include multiple cameras for receiving live images of surgical robot 25. In addition, a projector may be set up in a room with a projection screen in close proximity to surgical robot 25 such that the novice user may simultaneously see surgical robot 25 and an image of surgical robot 25 on the projection screen 90c. The projection screen 90c may display a live view of surgical robot 25 overlaid with augmented reality information, such as training information and/or commands. By viewing surgical robot 25 and the projection screen 90c simultaneously, the effect of a head-mounted IT interface 90a may be mimicked.

In an augmented reality embodiment in which the IT interface 90 may be implemented using a tablet computer 90b, the novice user may be present in the operating room with surgical robot 25 and may point a camera of the tablet computer 90b at surgical robot 25. The camera of the tablet computer 90b may then receive and process images of the surgical robot 25 to display the images of the surgical robot 25 on a display of the tablet computer 90b. As a result, an augmented reality view of surgical robot 25 is provided wherein the images of surgical robot 25 is overlaid with augmented reality information, such as training information and/or commands.

In still another augmented reality embodiment, IT interface 90 may be implemented as a projector system that may be used to project images onto surgical robot 25. For example, the projector system may include cameras for receiving images of surgical robot 25 from which a pose of surgical robot 25 is determined either in real time, such as by depth cameras or projection matching. Images from a database of objects may be used in conjunction with the received images to compute the pose of surgical robot 25 and to thereby provide for projection of objects by a projector of the projector system onto surgical robot 25.

In still another embodiment, IT interface 90 may be configured to present images to the user via both VR and AR. For example, a virtual surgical robot may be digitally created and displayed to the user via wearable device 90a, and sensors detecting movement of the user may then be used to update the images and allow the user to interact with the virtual surgical robot. Graphics and other images may be superimposed over the virtual surgical robot and presented to the view via wearable device 90a.

Regardless of the particular implementation, IT interface 90 may be a smart interface device configured to generate and process images on its own. Alternatively, IT interface 90 operates in conjunction with a separate computing device, such as computing device 95, to generate and process images to be displayed by IT interface 90. For example, a head-mounted IT interface device (not shown) may have a built-in computer capable of generating and processing images to be displayed by the head-mounted IT interface device, while a screen, such as a projection screen 90c or computer monitor (not shown), used for displaying AR or VR images would need a separate computing device to generate and process images to be displayed on the screen. Thus, in some embodiments, IT interface 90 and computing device 95 may be combined into a single device, while in other embodiments IT interface 90 and computing device 95 are separate devices.

Controller 60 is connected to and configured to control the operations of surgical robot 25 and any of IT interface 90. In an embodiment, console 80 is connected to surgical robot 25 and/or at least one IT interface 90 either directly or via a network (not shown). Controller 60 may be integrated into console 80, or may be a separate, stand-alone device connected to console 80 and surgical robot 25 via robotic base 18.

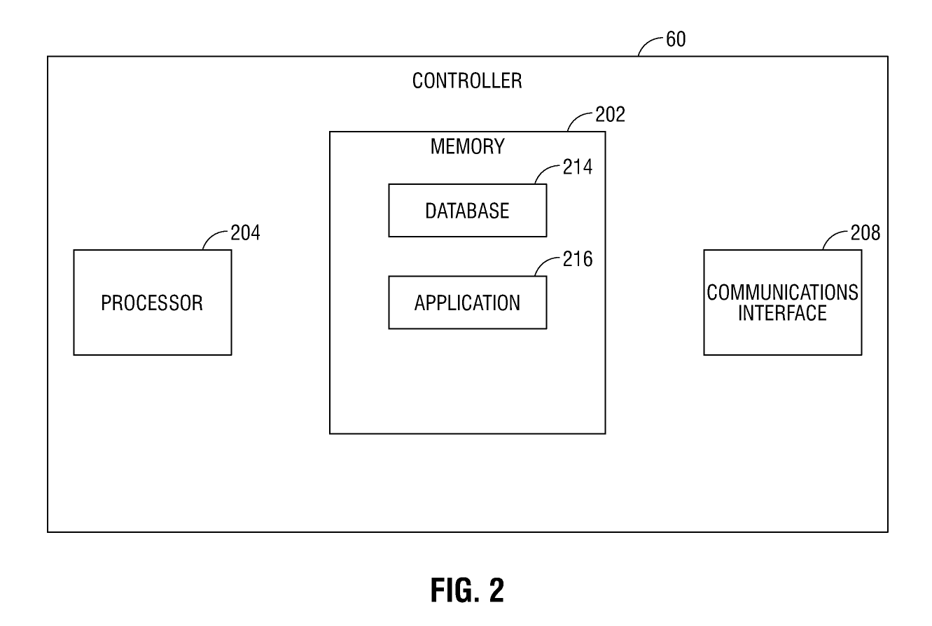

Turning now to FIG. 2, controller 60 may include memory 202, processor 204, and/or communications interface 206. Memory 202 includes any non-transitory computer-readable storage media for storing data and/or software that is executable by processor 204 and which controls the operation of controller 60.

Memory 202 may store an application 216 and/or database 214. Application 216 may, when executed by processor 204, cause at least one IT interface 90 to present images, such as virtual and/or augmented reality images, as described further below. Database 214 stores augmented reality training instructions, such as commands, images, videos, demonstrations, etc. Communications interface 206 may be a network interface configured to connect to a network connected to at least one IT interface 90, such as a local area network (LAN) consisting of a wired network and/or a wireless network, a wide area network (WAN), a wireless mobile network, a BLUETOOTH® network, and/or the internet. Additionally or alternatively, communications interface 206 may be a direct connection to at least one IT interface 90.

As noted above, virtual reality or augmented reality interfaces may be employed in providing user interaction with either a virtual surgical robot or with physical surgical robot 25 or a physical model for demonstrations. Selection of which interface to use may depend on the particular goal of the demonstration. For example, the virtual reality interface permits use with the virtual surgical robot. Thus, the virtual reality interface may be used to provide the user with virtual hands-on interaction, such as for training or high-level familiarity with surgical robot 25. Additionally, as a physical surgical robot is not necessary for use with a virtual reality interface, the virtual reality interface may be desirable in instances in which space may be an issue or in which it may not be feasible to access or place the physical surgical robot 25 at a particular location. For instances in which interaction with a physical surgical robot may be desired, the augmented reality interface may be implemented where the augmented reality interface supplements the physical surgical robot 25 with particular information either displayed thereon or in a display showing an image of the physical surgical robot 25. Thus, the user may be able to familiarize himself or herself with surgical robot 25 with physical interaction. Each of these embodiments will now be discussed in further detail separately below.

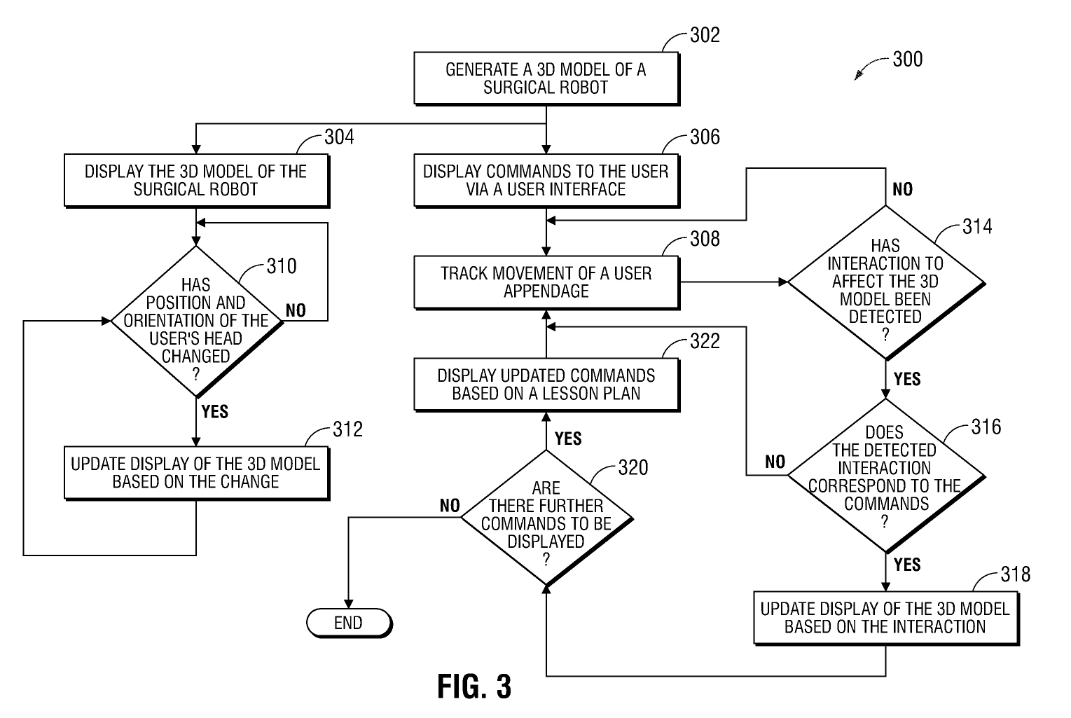

FIG. 3 is a flowchart of an exemplary method for using a virtual reality interface in training a user of a surgical robot, according to an embodiment of the present disclosure. The method of FIG. 3 may be performed using, for example, any one of IT interfaces 90 and computing device 95 of system 100 shown in FIG. 1. As noted above, IT interface 90 and computing device 95 may be separate devices or a single, combined device. For illustrative purposes in the examples provided below, an embodiment will be described wherein IT interface 90 is a head-mounted VR interface device (e.g., 90a) with a built-in computer capable of generating and processing its own images. However, any IT interface 90 may be used in the method of FIG. 3 without departing from the principles of the present disclosure.

Using the head-mounted VR interface device 90a, the user is presented with a view of a virtual surgical robot, based on designs and/or image data of an actual surgical robot 25. As described below, the user may virtually interact with the virtual surgical robot displayed by the VR interface device. The VR interface device is able to track movements of the user's head and other appendages, and based on such movements, may update the displayed view of the virtual surgical robot and determine whether a particular movement corresponds to an interaction with the virtual surgical robot.

Starting at step 302, IT interface 90 receives model data of surgical robot 25. The model data may include image data of an actual surgical robot 25, and/or a computer-generated model of a digital surgical robot similar to an actual surgical robot 25. IT interface 90 may use the model data to generate a 3D model of the digital surgical robot which will be used during the interactive training and with which the user will virtually interact. Thereafter, at step 304, IT interface 90 displays a view of the 3D model of the surgical robot. The view of the 3D model may be displayed in such a way that the user may view different angles and orientations of the 3D model by moving the user's head, rotating in place, and/or moving about.

IT interface 90 continually samples a position and an orientation of the user's head, arms, legs, hands, etc. (collectively referred to hereinafter as an “appendage”) as the user moves, in an embodiment. In this regard, sensors of IT interface 90, such as motion detection sensors, gyroscopes, cameras, etc. may collect data about the position and orientation of the user's head while the user is using IT interface 90. In particular, sensors connected to the user's head, hands, arms, or other relevant body parts to track movement, position, and orientation of such appendages. By tracking the movement of the appendage of the user, IT interface 90 may detect that the user performs a particular action, and/or may display different views of the 3D model and/or different angles and rotations of the 3D model.

By sampling the position and orientation of the user's head, IT interface 90 may determine, at step 310, whether the position and orientation of the user's head has changed. If IT interface 90 determines that the position and orientation of the user's head has changed, IT interface 90 may update, at step 312, the displayed view of the 3D model based on the detected change in the position and orientation of the user's head. For example, the user may turn his/her head to cause the displayed view of the 3D model of the digital surgical robot to be changed, e.g., rotated in a particular direction. Similarly, the user may move in a particular direction, such as by walking, leaning, standing up, crouching down, etc., to cause the displayed view of the surgical robot to be changed correspondingly. However, if IT interface 90 determines that the position and orientation of the user's head has not changed, the method iterates at step 310 so that IT interface 90 may keep sampling the position and orientation of the user's head to monitor for any subsequent changes.

Concurrently with the performance of steps 304, 310, and 312, IT interface 90 may receive a lesson plan and may generate commands based on the lesson plan. According to an embodiment, the lesson plan is preloaded into IT interface 90 to thereby provide a computer-guided experience from an online automated instruction system. In another embodiment, a portion of the lesson plan is preloaded into IT interface 90; however, other portions of the lesson plan may be provided by another source, such as a live source including a human mentor or trainer, or by another computer. At step 306, IT interface 90 displays the commands. The commands may be displayed as an overlay over the displayed view of the 3D model of the digital surgical robot. Alternatively, the commands may be displayed on an instruction panel separate from the view of the 3D model of the digital surgical robot. As noted above, the commands may be textual, graphical, and/or audio commands. The commands may also include demonstrative views of the 3D model of the digital surgical robot. For example, if the user is instructed to move a particular component, such as robotic arm 20, or connect a particular component to the surgical robot, the commands may illustrate the desired operation via a demonstrative view of the 3D model of the surgical robot.

Next, at step 308, IT interface 90 samples a position and an orientation of a user appendage as the user moves. By tracking the movement of the appendage of the user, IT interface 90 may detect that the user has performed a particular action. Based on the tracked movement of the user appendage, at step 314, IT interface 90 then detects whether an interaction with the 3D model of the digital surgical robot has occurred. If IT interface 90 detects that an interaction has been performed, the method proceeds to step 316. If IT interface 90 detects that an interaction has not been performed, the method returns to step 308, and IT interface 90 continues to track the movement of the appendage of the user to monitor for subsequent interactions.

At step 316, IT interface 90 determines whether the interaction corresponds to the commands. For example, IT interface 90 may determine, based on the tracked movement of the appendage of the user that a particular movement has been performed, and then determines whether this movement corresponds with the currently displayed commands. Thus, when the user successfully performs an interaction with the 3D model of the digital surgical robot as instructed by the commands, IT interface 90 determines that the command has been fulfilled. In another embodiment, IT interface 90 may indicate to the trainer whether or not the interaction corresponds to the commands. If so, at step 318, IT interface 90 updates the displayed view of the 3D model of the surgical robot based on the interaction between the appendage of the user and the virtual surgical robot. For example, when IT interface 90 determines that the user has performed a particular interaction with the digital surgical robot, such as moving a particular robotic arm 20, IT interface 90 updates the displayed view of the 3D model of the digital surgical robot based on the interaction. However, if, at step 316, the interaction does not correspond to the commands, the method returns to step 308, and IT interface 90 continues to track the movement of the appendage of the user to monitor for subsequent interactions. In another embodiment, further notification or communication from a trainer to the user may be provided indicating a suggested corrective action or further guidance, either via an updated display or an audible sound.

After the display is updated at step 318, a determination is made, at step 320, as to whether there are further commands to be displayed. If there are further commands to be displayed, the lesson is not complete, and the method proceeds to step 322 to display updated commands based on the lesson plan. However, if it is determined that there are no further commands to be displayed, the lesson is complete, and the method ends.

After the lesson has been completed, and/or at various intervals during the lesson, such as after the completion of a particular command, in addition to displaying the updated commands based on the lesson plan, IT interface 90 may further display a score to indicate how well the user's interaction corresponded with the commands. For example, the user may be given a percentage score based on a set of metrics. The set of metrics may include the time it took the user to perform the interaction, whether the user performed the interaction correctly the first time or whether the user, for example, moved robotic arm 20 incorrectly before moving it correctly, whether the user used the correct amount of force in performing the interaction, as opposed to too much or too little, etc. By scoring the user's performance of the commands included in the lesson plan, the user may be given a grade for each task performed. Additionally, the user's score may be compared with other users, and/or the user may be given an award for achieving a high score during training.

As noted above, interaction with surgical robot 25 may be performed using augmented reality. In an embodiment, by using the head-mounted AR interface device, the user may view a physical surgical robot, which may be either surgical robot 25 or a demonstrative model representing surgical robot 25 (collectively referred to as “physical model”), and AR interface device may display information and/or commands as overlays over the user's view of the physical model. As described below, the user may interact with the physical model and the AR interface device is able to track movements of the user's head and other appendages, and based on such movements, may update the displayed information and/or commands and determine whether a particular movement corresponds to an interaction with the physical model.

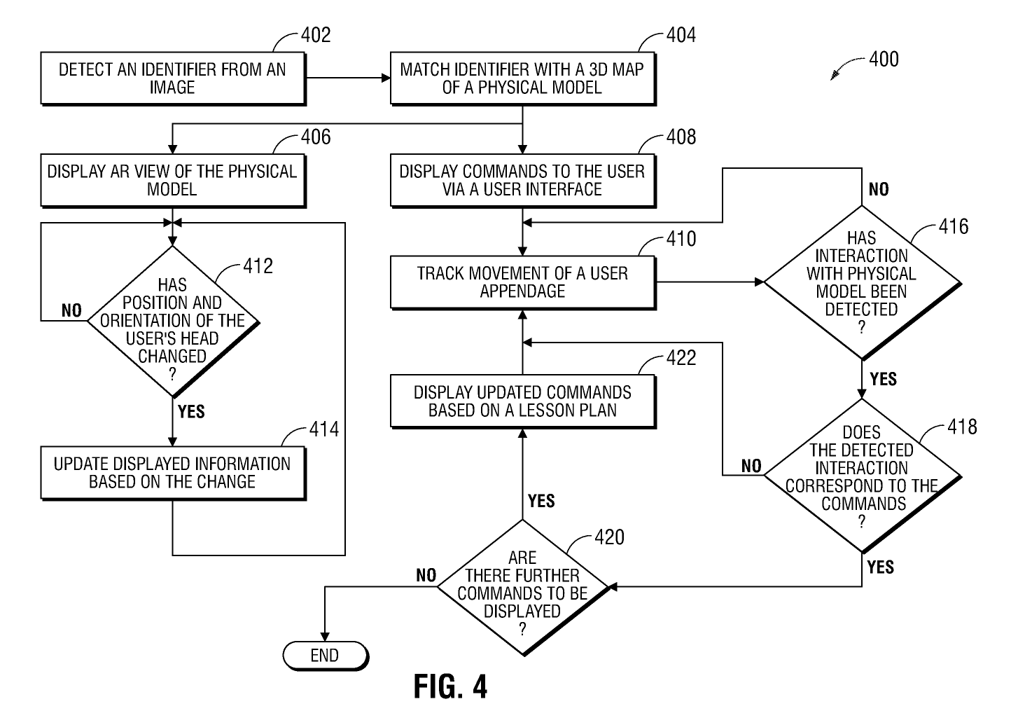

In this regard, turning now to FIG. 4, another example method for using an augmented reality interface in training a user of the physical model is provided. The method of FIG. 4 may be performed using, for example, IT interface 90 and computing device 95 of system 100 shown in FIG. 1. As noted above, IT interface 90 and computing device 95 may be separate devices or a single, combined device. For illustrative purposes in the examples provided below, here, an embodiment of method 400 will be described wherein IT interface 90 is a head-mounted AR interface device with a built-in computer capable of generating and processing its own images. However, any IT interface 90 may be used in the method of FIG. 4 without departing from the principles of the present disclosure.

Starting at step 402, an identifier is detected from images received from a camera. For example, in an embodiment, IT interface 90 receives images of the physical model, which may be collected by one or more cameras positioned about the room in which the physical model is located, by one or more cameras connected to the AR interface device, and the like. The physical model may be surgical robot 25, a miniature version of a surgical robot, a model having a general shape of surgical robot 25, and the like. The identifier may be one or more markers, patterns, icons, alphanumeric codes, symbols, objects, a shape, surface geometry, colors, infrared reflectors or emitters or other unique identifier or combination of identifiers that can be detected from the images using image processing techniques.

At step 404, the identifier detected from the images is matched with a three-dimensional (3D) surface geometry map of the physical model. In an embodiment, the 3D surface geometry map of the physical model may be stored in memory 202, for example, in database 216, and correspondence is made between the 3D surface geometry map of the physical model and the identifier. The result is used by IT interface 90 to determine where to display overlay information and/or commands.

At step 406, IT interface 90 displays an augmented reality view of the physical model. For example, IT interface 90 may display various information panels directed at specific parts or features of the physical model. The information may be displayed as an overlay over the user's view of the physical model. In an embodiment in which the physical model is a model having a general shape of surgical robot 25, a virtual image of surgical robot 25 may be displayed as an overlay over the user's view of the physical model and information may be superimposed on the user's view of the physical model. In order to properly display overlaid information over the user's view of the physical model, a determination is continuously made as to whether the user's head has changed position relative to the physical model at step 412. For example, by sampling the position and orientation of the user's head, IT interface 90 may determine, whether the position and orientation of the user's head has changed. If IT interface 90 determines that the position and orientation of the user's head has changed, IT interface 90 may update, at step 414, the displayed augmented reality view of the physical model (for example, the information relating to the physical model) based on the detected change in the position and orientation of the user's head. For example, the user may turn his/her head or move positions relative to surgical robot 25 to cause the displayed view of the overlaid information to be changed, e.g., rotated in a particular direction. Similarly, the user may move in a particular direction, such as by walking, leaning, standing up, crouching down, etc., to cause the displayed view of the overlaid information relative to the physical model to be changed correspondingly. However, if IT interface 90 determines that the position and orientation of the user's head has not changed, the method iterates at step 412 so that IT interface 90 may keep sampling the position and orientation of the user's head to monitor for any subsequent changes.

Thereafter, or concurrently therewith, IT interface 90 may receive a lesson plan and may generate commands based on the lesson plan, which may be entirely preloaded into IT interface 90 or partially preloaded into IT interface 90 and supplemented from other sources. The lesson plan may include a series of instructions for the user to follow, which may include interactions between the user and the physical model presented via IT interface 90. In an embodiment, the lesson plan may be a series of lessons set up such that the user may practice interacting with the physical model until certain goals are complete. Once completed, another lesson plan in the series of lessons may be presented.

In this regard, at step 408, which may be performed concurrently with steps 406, 412, and/or 414, IT interface 90 displays commands to the user. In an embodiment, the commands may be displayed in a similar manner as the information displayed in step 406, such as an overlay over the user's view of the physical model as viewed via IT interface 90. Alternatively, the commands may be displayed in an instruction panel separate from the user's view of the physical model. While the commands may be displayed as textual or graphical representations, it will be appreciated that one or more of the commands or portions of the commands may be provided as audio and/or tactile cues. In an embodiment, the commands may also include demonstrative views based on the physical model. For example, if the user is instructed to move a particular component, such as robotic arm 20, or connect a particular component to the surgical robot, the commands may illustrate the desired operation via a demonstrative view of a 3D model of the surgical robot superimposed upon the physical model.

Next, at step 410, IT interface 90 samples a position and an orientation of the user's head, arms, legs, hands, etc. (collectively referred to hereinafter as an “appendage”) as the user moves. For example, IT interface 90 may include sensors, such as motion detection sensors, gyroscopes, cameras, etc. which may collect data about the position and orientation of the user's head while the user is using IT interface 90. IT interface 90 may include sensors connected to the user's head, hands, arms, or other relevant body parts to track movement, position, and orientation of such appendages. By tracking the movement of the appendage of the user, IT interface 90 may detect that the user performs a particular action.

At step 416, IT interface 90 detects whether an interaction with the physical model has occurred based on the tracked movement of an appendage of the user. Alternatively, or in addition, IT interface 90 may receive data from the physical model that an interaction has been performed with the physical model, such as the movement of a particular robotic arm 20 and/or connection of a particular component. If IT interface 90 detects or receives data that an interaction has been performed, processing proceeds to step 418. If IT interface 90 detects that a particular interaction has not been performed, processing returns to step 410, where IT interface 90 continues to track the movement of the appendage of the user to monitor for subsequent interactions.

IT interface 90 further determines, at step 418, whether the interaction corresponds to the commands. For example, in an embodiment in which a command includes moving a robotic arm of the physical model to a particular location, IT interface 90 may determine or receive data from the physical model that the movement has been completed, and would then determine that the interaction corresponds to the currently displayed command. In another embodiment, IT interface 90 may indicate to the trainer whether or not the interaction corresponds to the commands. Alternatively, or in addition, IT interface 90 may determine, based on the tracked movement of the appendage of the user that a particular movement has been performed, and then determines whether this movement corresponds with the currently displayed commands. For example, when the user successfully performs an interaction with the physical model as instructed by the commands, IT interface 90 determines that the command has been fulfilled. However, if IT interface 90 determines that the particular movement does not correspond with the currently displayed commands, the method returns to step 410, and IT interface 90 continues to track the movement of the appendage of the user to monitor for subsequent interactions. In another embodiment, further notification or communication from a trainer to the user may be provided indicating a suggested corrective action or further guidance, either via an updated display or an audible sound.

At step 420, it is determined whether there are further commands to be displayed. If there are further commands to be displayed, the lesson is not complete, and the method proceeds to step 422 to display updated commands based on the lesson plan. However, if it is determined that there are no further commands to be displayed, the lesson is complete, and the method ends.

At step 422, IT interface 90 displays updated commands based on the lesson plan. It will be appreciated that in addition to displaying the updated commands based on the lesson plan, IT interface 90 may further display a score to indicate how well the user's interaction corresponded with the commands. For example, the user may be given a percentage score based on a set of metrics. The set of metrics may include the time it took the user to perform the interaction, whether the user performed the interaction correctly the first time or whether the user, for example, moved robotic arm 20 incorrectly before moving it correctly, whether the user used the correct amount of force in performing the interaction, as opposed to too much or too little, etc. By scoring the user's performance of the commands included in the lesson plan, the user may be given a grade for each task performed. Additionally, the user's score may be compared with other users, and/or the user may be given an award for achieving a high score during training.

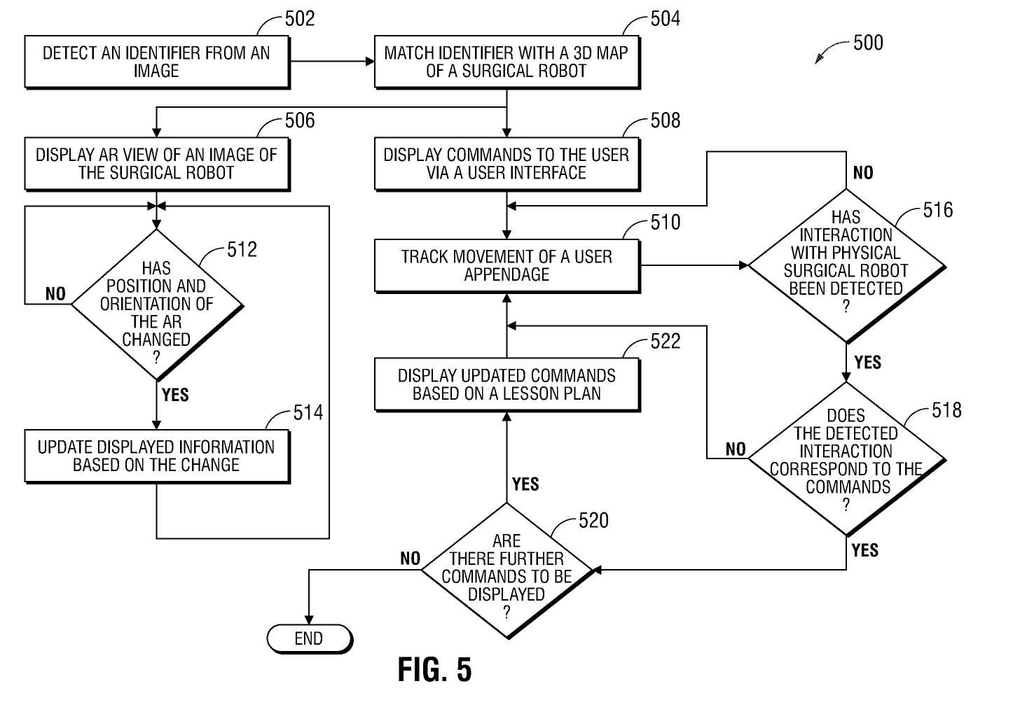

In another embodiment, it is also envisioned that, instead of using a head-mounted AR interface device, the user views a live view of surgical robot 25 on IT interface 90b or 90c, such as a portable electronic device such as a tablet, smartphone, and/or camera/projector/projection screen system, located nearby surgical robot 25 and the instructions and/or commands may likewise be displayed as overlays over the live view of surgical robot 25. For example, turning now to FIG. 5, a method 500 for using an augmented reality interface in training a user of a surgical robot in accordance with another embodiment is provided. The method of FIG. 5 may be performed using, for example, IT interface 90 and computing device 95 of system 100 shown in FIG. 1. As noted above, IT interface 90 and computing device 95 may be separate devices or a single, combined device. Here, an embodiment of method 500 will be described wherein IT interface 90 is a portable electronic device with a built-in computer capable of generating and processing its own images. However, any IT interface 90 may be used in the method of FIG. 5 without departing from the principles of the present disclosure.

Starting at step 502, an identifier is detected from images. For example, in an embodiment, IT interface 90 receives images of surgical robot 25, which may be collected by a camera included as part of the portable electronic device directed at surgical robot 25, by one or more cameras connected to IT interface device 90, and the like, and the identifier, which may be similar to the identifier described above for step 402 in method 400, is detected from the images. The detected identifier is matched with a three-dimensional (3D) surface geometry map of surgical robot 25 at step 504, and the result may be used by IT interface 90 to determine where to display overlay information and/or commands, and whether user interactions with surgical robot 25 are in accordance with displayed commands.

At step 506, IT interface 90 displays an augmented reality view of the image of surgical robot 25. For example, IT interface 90 may display various information panels overlaid on to specific parts or features of the displayed image of surgical robot 25. The information may be displayed as an overlay over the user's view of surgical robot 25 on a display screen of IT interface 90. In embodiments in which IT interface 90 is a smartphone or tablet 90b, in order to properly display overlaid information over the displayed image of surgical robot 25, a determination is continuously made as to whether the location of IT interface 90 (for example, portable electronic device) has changed position relative to surgical robot 25 at step 512. In an embodiment, by sampling the position and orientation of IT interface 90, a determination may be made as to whether the position and orientation of the IT interface 90 has changed. If the position and orientation of IT interface 90 has changed, IT interface 90 may update, at step 514, the displayed information relating to surgical robot 25 based on the detected change in the position and orientation of IT interface 90. IT interface 90 may be turned or moved relative to surgical robot 25 to cause the displayed image of both surgical robot 25 and the overlaid information to be changed, e.g., rotated in a particular direction. If IT interface 90 determines that its position and orientation has not changed, the method iterates at step 512 so that IT interface 90 may keep sampling its position and orientation to monitor for any subsequent changes.

No matter the particular implementation of IT interface 90, IT interface 90 may receive a lesson plan and may generate commands based on the lesson plan, which may be entirely preloaded into IT interface 90 or partially preloaded into IT interface 90 and supplemented from other sources. The lesson plan may include a series of instructions for the user to follow, which may include interactions between the user and surgical robot 25 presented via IT interface 90. In an embodiment, the lesson plan may be a series of lessons set up such that the user may practice interacting with surgical robot 25 until certain goals are complete. Once completed, another lesson plan in the series of lessons may be presented.

In this regard, at step 508, which may be performed concurrently with steps 506, 512, and/or 514, IT interface 90 displays commands to the user. In an embodiment, the commands may be displayed in a similar manner as the information displayed in step 506, such as an overlay over the displayed image of surgical robot 25 as viewed via IT interface 90. Alternatively, the commands may be displayed in an instruction panel separate from the displayed image of surgical robot 25. While the commands may be displayed as textual or graphical representations, it will be appreciated that one or more of the commands or portions of the commands may be provided as audio and/or tactile cues. In an embodiment, the commands may also include demonstrative views based on surgical robot 25. For example, if the user is instructed to move a particular component, such as robotic arm 20, or connect a particular component to the surgical robot, the commands may illustrate the desired operation via a demonstrative view of a 3D model of the surgical robot superimposed upon the displayed image of surgical robot 25.

In an embodiment, at step 510, IT interface 90 samples a position and an orientation of the user's head, arms, legs, hands, etc. (collectively referred to hereinafter as an “appendage”) as the user moves. For example, IT interface 90 may communicate with sensors, such as motion detection sensors, gyroscopes, cameras, etc. which may collect data about the position and orientation of the user's appendages while the user is using IT interface 90. IT interface 90 may include sensors connected to the user's head, hands, arms, or other relevant body parts to track movement, position, and orientation of such appendages. By tracking the movement of the appendage of the user, IT interface 90 may detect that the user performs a particular action.

At step 516, IT interface 90 detects whether an interaction with surgical robot 25 has occurred based on the tracked movement of an appendage of the user. Alternatively, or in addition, IT interface 90 may receive data from surgical robot 25 that an interaction has been performed, such as the movement of a particular robotic arm 20 and/or connection of a particular component. If IT interface 90 determines or receives data that an interaction has been performed, processing proceeds to step 518. If IT interface 90 determines that a particular interaction has not been performed, processing returns to step 510, where IT interface 90 continues to track the movement of the appendage of the user to monitor for subsequent interactions.

IT interface 90 further determines, at step 518, whether the interaction corresponds to the commands. For example, in an embodiment in which a command includes moving a robotic arm of surgical robot 25 to a particular location, IT interface 90 may determine or receive data from surgical robot 25 that the movement has been completed, and would then determine that the interaction corresponds to the currently displayed command. In another embodiment, IT interface 90 may indicate to the trainer whether or not the interaction corresponds to the commands. Alternatively, or in addition, IT interface 90 may determine, based on the tracked movement of the appendage of the user that a particular movement has been performed, and then determines whether this movement corresponds with the currently displayed commands. For example, when the user successfully performs an interaction with surgical robot 25 as instructed by the commands, IT interface 90 determines that the command has been fulfilled. However, if IT interface 90 determines that the particular movement does not correspond with the currently displayed commands, the method returns to step 510, and IT interface 90 continues to track the movement of the appendage of the user to monitor for subsequent interactions. In another embodiment, further notification or communication from a trainer to the user may be provided indicating a suggested corrective action or further guidance, either via an updated display or an audible sound.

At step 520, it is determined whether there are further commands to be displayed. If there are further commands to be displayed, the lesson is not complete, and the method proceeds to step 522 to display updated commands based on the lesson plan. However, if it is determined that there are no further commands to be displayed, the lesson is complete, and the method ends.

At step 522, IT interface 90 displays updated commands based on the lesson plan and may be performed in a manner similar to that described above with respect to step 522 of method 500.

The systems described herein may also utilize one or more controllers to receive various information and transform the received information to generate an output. The controller may include any type of computing device, computational circuit, or any type of processor or processing circuit capable of executing a series of instructions that are stored in a memory. The controller may include multiple processors and/or multicore central processing units (CPUs) and may include any type of processor, such as a microprocessor, digital signal processor, microcontroller, programmable logic device (PLD), field programmable gate array (FPGA), or the like. The controller may also include a memory to store data and/or instructions that, when executed by the one or more processors, causes the one or more processors to perform one or more methods and/or algorithms.

Any of the herein described methods, programs, algorithms or codes may be converted to, or expressed in, a programming language or computer program. The terms “programming language” and “computer program,” as used herein, each include any language used to specify instructions to a computer, and include (but is not limited to) the following languages and their derivatives: Assembler, Basic, Batch files, BCPL, C, C+, C++, Delphi, Fortran, Java, JavaScript, machine code, operating system command languages, Pascal, Perl, PL1, scripting languages, Visual Basic, metalanguages which themselves specify programs, and all first, second, third, fourth, fifth, or further generation computer languages. Also included are database and other data schemas, and any other meta-languages. No distinction is made between languages which are interpreted, compiled, or use both compiled and interpreted approaches. No distinction is made between compiled and source versions of a program. Thus, reference to a program, where the programming language could exist in more than one state (such as source, compiled, object, or linked) is a reference to any and all such states. Reference to a program may encompass the actual instructions and/or the intent of those instructions.

Any of the herein described methods, programs, algorithms or codes may be contained on one or more machine-readable media or memory. The term “memory” may include a mechanism that provides (e.g., stores and/or transmits) information in a form readable by a machine such a processor, computer, or a digital processing device. For example, a memory may include a read only memory (ROM), random access memory (RAM), magnetic disk storage media, optical storage media, flash memory devices, or any other volatile or non-volatile memory storage device. Code or instructions contained thereon can be represented by carrier wave signals, infrared signals, digital signals, and by other like signals.

While several embodiments of the disclosure have been shown in the drawings, it is not intended that the disclosure be limited thereto, as it is intended that the disclosure be as broad in scope as the art will allow and that the specification be read likewise. Therefore, the above description should not be construed as limiting, but merely as exemplifications of particular embodiments. Those skilled in the art will envision other modifications within the scope and spirit of the claims appended hereto.

1. A method of training a user of a robotic surgical system including a surgical robot using a virtual reality interface, the method comprising:
generating a three-dimensional (3D) model of the surgical robot;
displaying a view of the 3D model of the surgical robot using the virtual reality interface;
continuously sampling a position and orientation of feature of the user as the feature of the user is moved; and
updating the displayed view of the 3D model of the surgical robot based on the sampled position and orientation of the feature of the user.

2. The method of claim 1, further comprising:
tracking movement of an appendage of the user;
determining an interaction with the 3D model of the surgical robot based on the tracked movement of the appendage of the user; and
updating the displayed view of the 3D model of the surgical robot based on the interaction.

3. The method of claim 1, further comprising displaying commands based on a lesson plan using the virtual reality interface.

4. The method of claim 3, further comprising:
determining whether the interaction corresponds to the commands; and
displaying updated commands based on the lesson plan when it is determined that the interaction corresponds to the commands.

5. The method of claim 3, wherein the displaying commands includes displaying commands instructing the user to perform a movement to interact with the 3D model of the surgical robot.

6. (canceled)

7. The method of claim 4, further comprising displaying a score based on objective measures used to assess a user performance based on the interactions instructed by the commands.

8. The method of claim 1, wherein the displaying includes displaying the view of the 3D model using a head-mounted virtual reality display.

9. The method of claim 1, wherein the displaying includes projecting the view of the 3D model using a projector system.

10-27. (canceled)

28. A method of training a user of a robotic surgical system including a surgical robot using an augmented reality interface including an augmented reality interface device, the method comprising:
detecting an identifier in an image including a physical model;
matching the identifier with a three-dimensional surface geometry map of a physical model representing the surgical robot;
displaying an augmented reality view of the physical model;
continuously sampling a position and orientation of a user's head relative to a location of the physical model; and
updating the displayed augmented reality view of the physical model based on the sampled position and orientation of the head of the user.

29. The method of claim 28, further comprising:
tracking movement of an appendage of the user;
determining an interaction with the physical model representing the surgical robot based on the tracked movement of the appendage of the user; and
updating the displayed augmented reality view of the physical model based on the interaction.

30. The method of claim 29, further comprising displaying commands based on a lesson plan using the augmented reality interface.

31. The method of claim 30, further comprising:
determining whether the interaction corresponds to the commands; and
displaying updated commands based on the lesson plan in response to a determination that the interaction corresponds to the commands.

32. The method of claim 30, wherein the displaying commands includes displaying commands instructing the user to perform a movement to interact with the physical model representing the surgical robot.

33-34. (canceled)

35. The method of claim 28, wherein the physical model is the surgical robot.

36. A method of training a user of a robotic surgical system including a surgical robot using an augmented reality interface including an augmented reality interface device, the method comprising:
detecting an identifier in an image including the surgical robot;
matching the identifier with a three-dimensional surface geometry map of the surgical robot;
displaying an augmented reality view of an image of the surgical robot;
continuously sampling a position and orientation of the augmented reality interface device relative to a location of the surgical robot; and
updating the displayed augmented reality view of the surgical robot based on the sampled position and orientation of the augmented reality interface device.

37. The method of claim 36, further comprising:
tracking movement of an appendage of the user;
determining an interaction with the surgical robot based on the tracked movement of the appendage of the user; and
updating the displayed augmented reality view of the surgical robot based on the interaction.

38. The method of claim 37, further comprising displaying commands based on a lesson plan using the augmented reality interface.

39. The method of claim 38, further comprising:
determining whether the interaction corresponds to the commands; and
displaying updated commands based on the lesson plan in response to a determination that the interaction corresponds to the commands.

40. The method of claim 38, wherein the displaying commands includes displaying commands instructing the user to perform a movement to interact with the surgical robot.

41. (canceled)

42. The method of claim 37, wherein the displaying includes displaying the augmented reality view of an image of the surgical robot using a tablet, smartphone, or projection screen.
